(19) United States
(12) Patent Application Publication
(10) Pub. No.: US 2026/0128160 A1
(43) Pub. Date: May 7, 2026

(54) SYSTEM FOR CATHETER AND SHADOW SUPPRESSION IN RENDERED 3D ECHOCARDIOGRAPHY

(71) Applicant: GE Precision Healthcare LLC, Waukesha, WI (US)

(72) Inventors: Hani Nozari MIRAR, Oslo (NO); Olivier GERARD, Oslo (NO)

(21) Appl. No.: 18/936,389

(22) Filed: Nov. 4, 2024

(51) Int. Cl. G16H 30/40; G06T 5/50; G06T 5/77; G06V 10/25

(52) U.S. Cl. CPC G16H 30/40; G06T 5/50; G06T 5/77; G06V 10/25; G06V 2201/034

(57) ABSTRACT

A system may acquire a first medical image, of a region of interest of a subject, in which one or more medical instruments are present in the region of interest of the subject. The system may determine one or more regions of the first medical image corresponding to the one or more medical instruments. The system may generate a second medical image in which the one or more medical instruments are absent using first medical image data of the first medical image external to the one or more regions and inpainted data internal to the one or more regions. The system may display the second medical image. The system may display one or more visual indicators that indicate a respective positions and/or directions of the one or more medical instruments in the second medical image.

### TECHNICAL FIELD

The present disclosure relates to a system for acquiring a medical image, of a region of interest of a subject, in which a medical instrument is present in the region of interest, and generating a medical image of the region of interest in which the medical instrument is absent.

### BACKGROUND

During an interventional procedure, a clinician might navigate a medical instrument through a region of interest of a subject. For instance, during a cardiac procedure, a clinician may navigate a medical instrument (e.g., a catheter) through a heart of a subject in order to ablate tissue, insert a mitral valve clip, close a left atrial appendage, deliver a stent, remove a thrombus, analyze cardiac function, or the like. Also, during the interventional procedure, a medical imaging system (e.g., an ultrasound imaging system) may acquire medical images of the region of interest and the medical instrument, and display the medical images to assist the clinician in navigating the medical instrument through the region of interest. In some cases, the display of the medical instrument in the medical images might obfuscate the medical images by occluding various underlying anatomical features of the region of interest. Moreover, the medical instrument may cause shadows in the medical images, which also might obfuscate the medical images. Accordingly, the clinician might not be able to ascertain anatomical features that are occluded by the medical instrument or the shadow of the medical instrument.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: acquire a first medical image, of a region of interest of a subject, in which a medical instrument is present in the region of interest of the subject; determine a region of the first medical image corresponding to the medical instrument; generate a second medical image in which the medical instrument is absent using first medical image data of the first medical image external to the region and inpainted data internal to the region; and display the second medical image.

In another aspect, a method may include acquiring a first medical image, of a region of interest of a subject, in which a medical instrument is present in the region of interest of the subject; determining a region of the first medical image corresponding to the medical instrument; generating a second medical image in which the medical instrument is absent using first medical image data of the first medical image external to the region and inpainted data internal to the region; and displaying the second medical image.

In yet another aspect, a non-transitory computer-readable medium may store instructions that, when executed by one or more processors, cause the one or more processors to: acquire a first medical image, of a region of interest of a subject, in which a medical instrument is present in the region of interest of the subject; determine a region of the first medical image corresponding to the medical instrument; generate a second medical image in which the medical instrument is absent using first medical image data of the first medical image external to the region and inpainted data internal to the region; and display the second medical image.

### BRIEF DESCRIPTION OF DRAWINGS

FIG. 1 is a diagram of an example system for generating a medical image in which a medical instrument is absent.

FIG. 2 is a diagram of example components of one or more devices of FIG. 1.

FIG. 3 is a diagram of example components of the medical imaging system of FIG. 1.

FIG. 4 is a diagram of example components of the tracking system of FIG. 1.

FIG. 5 is a flowchart of an example process for generating a medical image in which a medical instrument is absent.

FIGS. 6A and 6B are diagrams of medical images.

FIGS. 7A and 7B are diagrams of medical images.

FIG. 8 is a flowchart of an example process for generating a medical image in which a medical instrument is absent or present based on whether the display of the medical instrument is toggled on or toggled off.

### DETAILED DESCRIPTION

As addressed above, the display of a medical instrument and/or a shadow of a medical instrument in medical images might obfuscate the medical images by occluding various underlying anatomical features of the region of interest. Accordingly, a clinician might not be able to ascertain anatomical features that occluded by the medical instrument or the shadow of the medical instrument. In this way, the displayed medical images might not comprehensively depict the region of interest, which might inhibit the clinician's ability to quickly and readily assess the entire region of interest during an interventional procedure.

Some embodiments herein provide a system for generating medical images in which a medical instrument is absent from a depicted region of interest despite the medical instrument actually being physically present in the region of interest. In this way, some embodiments herein provide a technical improvement in the technical field of medical imaging by generating a more comprehensive and accurate medical image that is not obfuscated by the present of a medical instrument and a shadow of the medical instrument. Further, in this way, some embodiments herein provide a technical improvement to medical imaging systems and user interfaces of medical imaging systems by permitting the medical imaging systems to generate more comprehensive and visually informative medical images, and by permitting a clinician to selectively toggle the display of the medical instrument on and off during an interventional procedure.

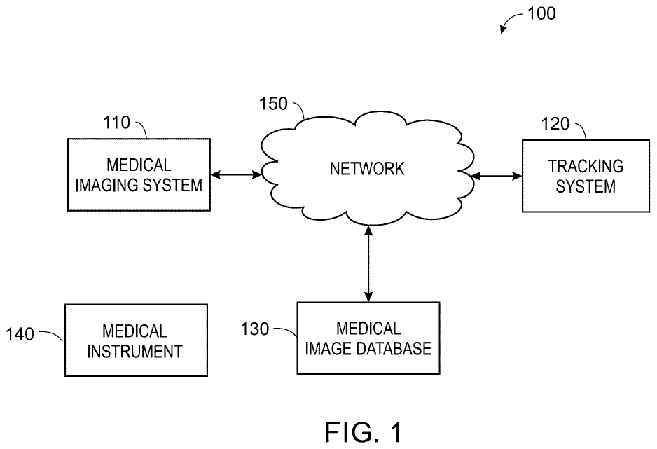

FIG. 1 is a diagram of an example system 100 for generating a medical image in which a medical instrument is absent. As shown in FIG. 1, the system 100 may include a medical imaging system 110, a tracking system 120, a medical image database 130, a medical instrument 140, and a network 150.

The medical imaging system 110 may be configured to acquire a first medical image, of a region of interest of a subject, in which a medical instrument is present in the region of interest of the subject; determine a region of the first medical image corresponding to the medical instrument; generate a second medical image in which the medical instrument is absent using first medical image data of the first medical image external to the region and inpainted data internal to the region; and display the second medical image. For example, the medical imaging system 110 may be an ultrasound system, a computed tomography (CT) system, a magnetic resonance imaging (MRI) system, an ultrasound system, an X-ray system, a positron emission tomography (PET) device, or the like.

The tracking system 120 may be configured to acquire tracking data of the medical instrument 140 located within the region of interest of the subject. For example, the tracking system 120 may be an electromagnetic tracking system, an optical tracking system, an acoustic tracking system, an inertial tracking system, an ultrasound tracking system, or the like.

The medical image database 130 may be configured to store medical images. For example, the medical image database 130 may be a cloud database, a hierarchical database, a network database, a centralized database, a picture archiving and communication system (PACS), or the like.

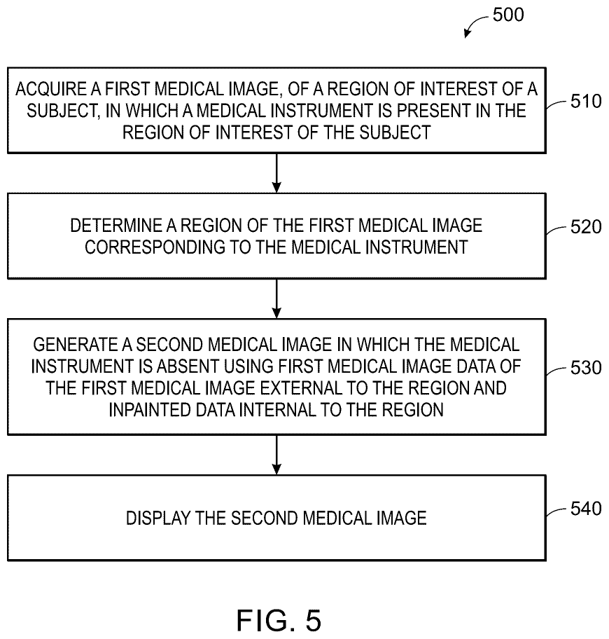

The medical instrument 140 may be any medical instrument that can be navigated through a region of interest of a subject. For example, the medical instrument 140 may be a catheter, a needle, a trocar, a cannula, or the like. The medical instrument 140 may be used for various interventional procedures involving the region of interest. For example, a catheter may be used for delivering a stent to an occluded blood vessel, inserting a mitral valve clip, closing a left atrial appendage, ablating tissue, analyzing cardiac function, removing a thrombus from an occluded blood vessel, or the like. Alternatively, the medical instrument 140 may be an implantable device that is to be implanted in the heart of the subject. For example, the implantable device may be a pacemaker, a stent, a defibrillator, a left ventricular assist device, a valve clip, or the like. Alternatively, the medical instrument 140 may be any object that can be navigated throughout the region of interest and/or tracked through the region of interest. One or more medical instruments 140 may be provided within and navigated through the region of interest. Accordingly, it should be understood that the embodiments herein are applicable to situations in which any number of medical instruments 140 are provided within and navigated through the region of interest. Restated, the medical imaging system 110 may perform one or more operations of FIG. 5 with respect to any number of medical instruments 140 in the medical image.

The network 150 may permit communication between the medical imaging system 110, the tracking system 120, and the medical image database130. For example, the network 150 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

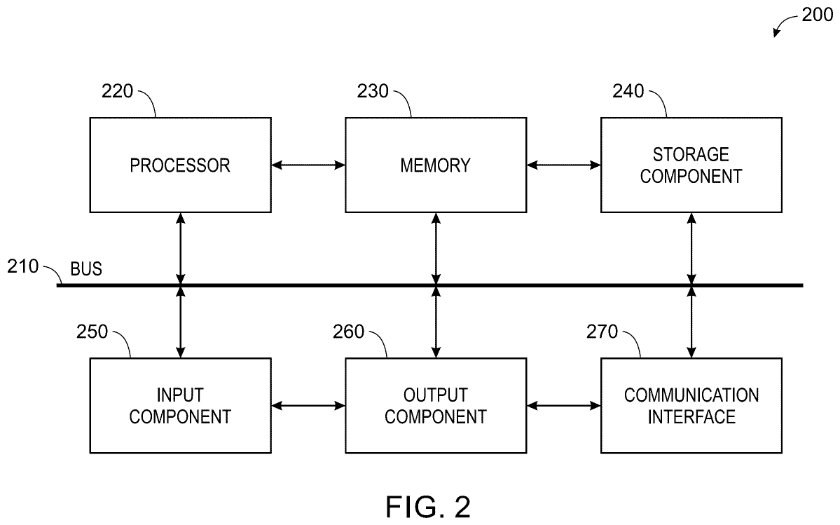

FIG. 2 is a diagram of example components of one or more devices 200 of FIG. 1. The device 200 may correspond to the medical imaging system 110, the tracking system 120, and/or the medical imaging database 130. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 220 may include one or more processors capable of being programmed to perform a function. The processor 220 may include one or more processors 220 configured to perform the operations described herein. For example, a single processor 220 may be configured to perform all of the operations described herein. Alternatively, multiple processors 220, collectively, may be configured to perform all of the operations described herein, and each of the multiple processors 220 may be configured to perform a subset of the operations descried herein. For example, a first processor 220 may perform a first subset of the operations described herein, a second processor 220 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other systems, such as via a wired connection, a wireless connection, or a combination of wired and wireless connections. The communication interface 270 may permit the device 200 to receive information from another system and/or provide information to another system. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another system via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components of the device 200 shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

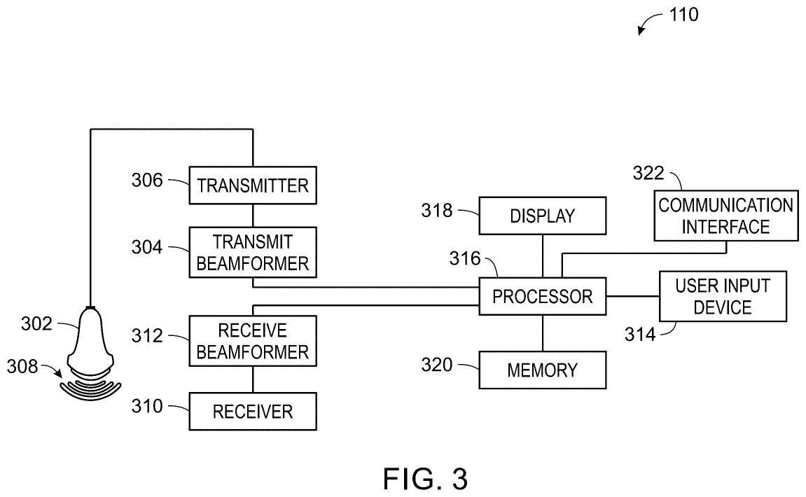

FIG. 3 is a diagram of example components of the medical imaging system of FIG. 1. As shown in FIG. 3, the medical imaging system 110 may include an ultrasound probe 302, a transmit beamformer 304, a transmitter 306, elements 308 a receiver 310, a receive beamformer 312, a user input device 314, a processor 316, a display 318, a memory 320, and a communication interface 322. The foregoing components may be connected via wired or wireless connections.

The ultrasound probe 302 may be configured to acquire ultrasound data of a region of interest of a subject. For example, the ultrasound probe 302 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 302 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The transmit beamformer 304 may be configured to apply delay times to electrical signals provided to the elements 308 to focus corresponding ultrasound signals at the region of interest. The transmitter 306 may be configured to transmit electrical signals to the elements 308 to drive the elements 308 to emit ultrasound signals towards the region of interest. The elements 308 may be configured to receive the electrical signals from the transmitter 306, convert the electrical signals into ultrasound signals, and emit the ultrasound signals towards the region of interest. The elements 308 may be configured to receive echo ultrasound signals that are back-scattered by the region of interest, convert the echo ultrasound signals into electrical signals, and provide the electrical signals to the receiver 310. The receiver 310 may be configured to receive electrical signals from the elements 308, and provide the electrical signals to the receive beamformer 312. The receive beamformer 312 may apply delay times to the electrical signals received from the elements 308.

The user input device 314 may be configured to receive a user input, and provide the user input to the processor 316. For example, the user input device 314 may be a touch screen display, a keyboard, a keypad, a mouse, a button, a switch, a microphone, or the like. Additionally, or alternatively, the user input device 314 may be configured to sense information. For example, the user input device 314 may sense information from an electro-magnetic positioning system, an inertial measurement system, an accelerometer, a gyroscope, an actuator, or the like.

The processor 316 may be configured to perform the operations as described herein. For example, the processor 316 may be a CPU, a GPU, an APU, a microprocessor, a microcontroller, a DSP, an FPGA, an ASIC, or another type of processing component. The processor 316 may be implemented in hardware, firmware, or a combination of hardware and software. The processor 316 may include one or more processors 316 configured to perform the operations described herein. For example, a single processor 316 may be configured to perform all of the operations described herein. Alternatively, multiple processors 316, collectively, may be configured to perform all of the operations described herein, and each of the multiple processors 316 may be configured to perform a subset of the operations descried herein. For example, a first processor 316 may perform a first subset of the operations described herein, a second processor 316 may be configured to perform a second subset of the operations described herein, etc.

The processor 316 may be configured to control the ultrasound probe 302 to acquire ultrasound data. The processor 316 may be configured to control which of the elements 308 are active, and control the shape of a beam emitted from the ultrasound probe 302. The processor 316 may generate ultrasound images for display. For example, the processor 316 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 318 may be configured to display information. For example, the display 318 may be a monitor, an LED display, a cathode ray tube, a projector display, a touchscreen, tablet computer, mobile phone, or the like. The display 318 may display ultrasound images based on the ultrasound data in real-time. For example, the display 318 may display the ultrasound images within one second, two seconds, five seconds, etc., of the ultrasound data being acquired by the ultrasound probe 302.

The memory 320 may be configured to store information and/or instructions for use by the processor 316. The memory 320 may be a non-transitory computer-readable medium. For example, the memory 320 may be a RAM, a ROM, and/or another type of dynamic or static storage device (e.g., a flash memory, a magnetic memory, and/or an optical memory) that stores information and/or instructions for use by the processor 316. The memory 320 may be configured to store instructions that, when executed by the processor 316, cause the processor 316 to perform the operations described herein.

The communication interface 322 may be configured to enable the processor 316 to communicate with other systems, such as via a wired connection, a wireless connection, or a combination of wired and wireless connections. For example, the communication interface 322 may include an Ethernet interface, an optical interface, a coaxial interface, an infrared interface, an RF interface, a USB interface, a Wi-Fi interface, a cellular network interface, or the like.

The number and arrangement of the components of the medical imaging system 110 shown in FIG. 3 are provided as an example. In practice, the medical imaging system 110 may include additional components, fewer components, different components, or differently arranged components than those shown in FIG. 3. Additionally, or alternatively, a set of components (e.g., one or more components) of the medical imaging system 110 may perform one or more functions described as being performed by another set of components of the medical imaging system 110.

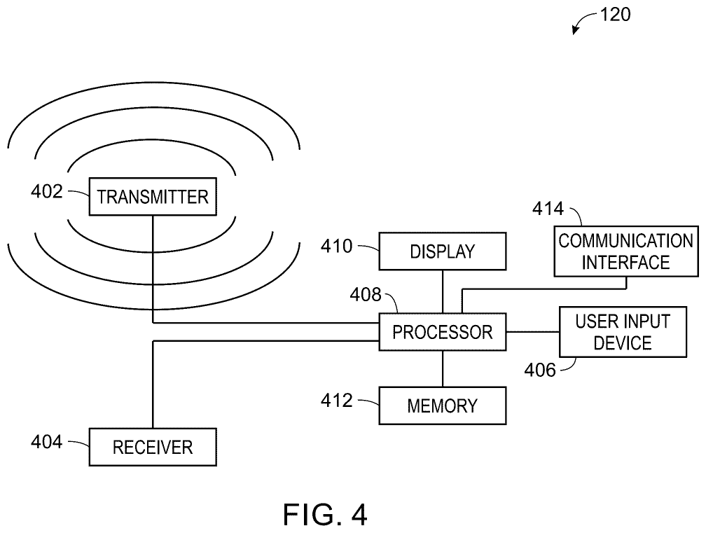

FIG. 4 is a diagram of example components of the tracking system of FIG. 1. As shown in FIG. 4, the tracking system 120 may include a transmitter 402, a receiver 404, a user input device 406, a processor 408, a display 410, a memory 412, and a communication interface 414.

The transmitter 402 may be configured to generate a magnetic field. The receiver 404 may be configured to output a signal in response to the magnetic field generated by the transmitter 402. The processor 408 may receive the output signal from the receiver 404, and acquire tracking data that identifies a position and/or an orientation of the receiver 404. According to an embodiment, the receiver 404 may be attached to the ultrasound probe 302 to track a position and/or an orientation of the ultrasound probe 302. Alternatively, the receiver 404 may be attached to the medical instrument 140 to track a position and/or an orientation of the medical instrument 140. Alternatively, the receiver 404 may be attached to the feature in the region of interest.

The user input device 406 may be configured to receive a user input, and provide the user input to the processor 408. For example, the user input device 406 may be a touch screen display, a keyboard, a keypad, a mouse, a button, a switch, a microphone, or the like. Additionally, or alternatively, the user input device 406 may be configured to sense information. For example, the user input device 406 may sense information from an electro-magnetic positioning system, an inertial measurement system, an accelerometer, a gyroscope, an actuator, or the like.

The processor 408 may be configured to perform the operations as described herein. For example, the processor 408 may be a CPU, a GPU, an APU, a microprocessor, a microcontroller, a DSP, an FPGA, an ASIC, or the like. The processor 408 may be implemented in hardware, firmware, or a combination of hardware and software. The processor 408 may include one or more processors 408 configured to perform the operations described herein. For example, a single processor 408 may be configured to perform all of the operations described herein. Alternatively, multiple processors 408, collectively, may be configured to perform all of the operations described herein, and each of the multiple processors 408 may be configured to perform a subset of the operations descried herein. For example, a first processor 408 may perform a first subset of the operations described herein, a second processor 408 may be configured to perform a second subset of the operations described herein, etc.

The processor 408 may be configured to control the transmitter 402 to acquire tracking data. The processor 408 may be configured to control excitations of the transmitter 402 to generate a magnetic field. The processor 408 may acquire tracking data based on controlling the transmitter 402.

The display 410 may be configured to display information. For example, the display 410 may be a monitor, an LED display, a cathode ray tube, a projector display, a touchscreen, tablet computer, mobile phone, or the like. The display 410 may display the tracking data in real-time. For example, the display 410 may display the tracking data within one second, two seconds, five seconds, etc., of the tracking data being acquired.

The memory 412 may be configured to store information and/or instructions for use by the processor 408. The memory 412 may be a non-transitory computer-readable medium. For example, the memory 412 may be a RAM, a ROM, a flash memory, a magnetic memory, an optical memory, or the like. The memory 412 may be configured to store instructions that, when executed by the processor 408, cause the processor 408 to perform the operations described herein.

The communication interface 414 may be configured to enable the processor 408 to communicate with other systems, such as via a wired connection, a wireless connection, or a combination of wired and wireless connections. For example, the communication interface 414 may include an Ethernet interface, an optical interface, a coaxial interface, an infrared interface, an RF interface, a USB interface, a Wi-Fi interface, a cellular network interface, or the like.

The number and arrangement of the components of the tracking system 120 shown in FIG. 4 are provided as an example. In practice, the tracking system 120 may include additional components, fewer components, different components, or differently arranged components than those shown in FIG. 4. Additionally, or alternatively, a set of components (e.g., one or more components) of the tracking system 120 may perform one or more functions described as being performed by another set of components of the tracking system 120.

Although FIG. 4 depicts the tracking system 120 as being an electromagnetic tracking system, it should be understood that the embodiments herein are applicable to other types of tracking systems, such as optical tracking systems, acoustic tracking systems, ultrasound tracking systems, AI-based tracking methods, or the like.

FIG. 5 is a flowchart of an example process 500 for generating a medical image in which a medical instrument is absent. According to an embodiment, the medical imaging system 110 may be configured to perform one or more operations of the process 500. Alternatively, another device may be configured to perform one or more operations of the process 500.

As shown in FIG. 5, the process 500 may include acquiring a first medical image, of a region of interest of a subject, in which a medical instrument is present in the region of interest of the subject (operation 510). For example, the medical imaging system 110 may acquire a first medical image, of a region of interest of a subject, in which the medical instrument 140 is present in the region of interest of the subject. According to an embodiment, the medical imaging system 110 may acquire the first medical image during an interventional procedure in which the medical instrument 140 is navigated through the region of interest of the subject. During the interventional procedure, one or more medical instruments 140 may be positioned within and navigated through the region of interest. Accordingly, the first medical image may depict the one or more medical instruments 140.

As further shown in FIG. 5, the process 500 may include determining a region of the first medical image corresponding to the medical instrument (operation 520). For example, the medical imaging system 110 may determine a region of the first medical image corresponding to the medical instrument.

According to an embodiment, the medical imaging system 110 may determine the region of the first medical image corresponding to the medical instrument 140 using previous medical images of the region of interest of the subject in which the medical instrument 140 is absent. The previous medical images may be medical images of the region of interest of the subject in which the medical instrument 140 is absent. According to an embodiment, the medical imaging system 110 may acquire the previous medical images in substantially real-time with the interventional procedure. For example, the medical imaging system 110 may acquire the previous medical images at the beginning of the interventional procedure, a threshold amount of time (e.g., 10 minutes, 30 minutes, etc.) before the interventional procedure, a threshold amount of time (e.g., 30 seconds, 1 minute, etc.) before the medical instrument 140 is inserted into the region of interest, or the like. Alternatively, the medical imaging system 110 may acquire the previous medical images from the medical image database 130. In this case, the previous medical images may have been acquired in non-real-time with the interventional procedure. For example, the previous medical images may have been acquired a day before the interventional procedure, a month before the interventional procedure, or the like. The previous medical images may be correlated with an event. For example, the previous medical images may be correlated with a cardiac cycle of the subject. Additionally, or alternatively, the previous medical images may be correlated with a timeframe. For example, the previous medical images may have respective timestamps that identify positions of the respective previous medical images in relation to a timeframe of a cardiac cycle. Additionally, or alternatively, the previous medical images may be correlated with bio-signal data. For example, the previous medical images may be correlated with electrocardiogram (ECG) data. The medical imaging system 110 may determine one or more particular previous medical images to which to compare to the first medical image to determine the region of the first medical image corresponding to the medical instrument 140. For example, the medical imaging system 110 may determine a timestamp of the first medical image that identifies a position of the first medical image in the cardiac cycle. Then, the medical imaging system 110 may determine a previous medical image that corresponds to the same position in the cardiac cycle. The medical imaging system 110 may compare the first medical image and one or more previous medical images to determine the region of the first medical image corresponding to the medical instrument 140. For example, the medical imaging system 110 may determine areas of the first medical image that are different than the one or more previous medical images, and determine the region of the first medical image based on the areas that are different.

According to an embodiment, the medical imaging system 110 may determine the region of the first medical image corresponding to the medical instrument 140 using tracking data from the tracking system 120. The tracking data may identify a position of the medical instrument 140. The medical imaging system 110 may use the tracking data to determine a position of the medical instrument 140 in the first medical image. Then, the medical imaging system 110 may determine the region of the first medical image corresponding to the medical instrument 140 based on the position of the medical instrument 140 in the first medical image.

According to an embodiment, the medical imaging system 110 may determine the region of the first medical image corresponding to the medical instrument 140 using an image processing technique. For example, the medical imaging system 110 may analyze the first medical image using an image processing technique (e.g., an object detection technique, an image segmentation technique, an edge detection technique, or the like), and determine the region of the first medical image corresponding to the medical instrument 140 based on analyzing the first medical image.

According to an embodiment, the medical imaging system 110 may determine the region of the first medical image corresponding to the medical instrument 140 using an artificial intelligence (AI) model. For example, the medical imaging system 110 may input the first medical image into an AI model (e.g., a convolutional neural network (CNN), a generative adversarial networks (GAN), a recurrent neural network (RNN), or the like), and determine the region of the first medical image corresponding to the medical instrument 140 based on an output of the AI model. In this case, the AI model may be trained using training data that identifies a position of the medical instrument 140 in a region of interest.

According to an embodiment, the medical imaging system 110 may determine one or more regions of the first medical image that respectively correspond to the one or more medical instruments 140. For example, if the first medical image depicts a single medical instrument 140, then the medical imaging system 110 may determine a single region that corresponds to the single medical instrument 140. Alternatively, if the first medical image depicts n medical instruments 140, then the medical imaging system 110 may determine n regions that respectively correspond to the n medical instruments 140.

As further shown in FIG. 5, the process 500 may include generating a second medical image in which the medical instrument is absent using first medical image data of the first medical image external to the region and inpainted data internal to the region (operation 530). For example, the medical imaging system 110 may generate a second medical image in which the medical instrument 140 is absent using first medical image data of the first medical image external to the region and inpainted data internal to the region. The medical imaging system 110 may generate the second medical image using first medical image data of the first medical image external to the region, and using inpainted data internal to the region. Restated, the second medical image may be the same as the first medical image external to the region, and may be different than the first medical image internal to the region. The inpainted data may be data that depicts the region of interest in which the medical instrument 140 is absent.

The medical imaging system 110 may generate a mask that delineates the region, and generate the second medical image including the first medical image data of the first medical image external to the region and inpainted data internal to the region, based on the first medical image and the mask. For example, the medical imaging system 110 may remove the first medical imaging data from the region based on the mask, and inpaint data internal to the region.

According to an embodiment, the medical imaging system 110 may generate the inpainted data using a previous medical image of the region of interest of the subject. For example, the medical imaging system 110 may determine previous medical image data that corresponds to the region, and inpaint the region using the previous medical image data. Alternatively, the medical imaging system 110 may generate the inpainted data using an inpainting technique. Alternatively, the medical imaging system 110 may generate the inpainted data using an AI model. For example, the medical imaging system 110 may input the first medical image in which the region is identified into an AI model, and generate the inpainted data based on an output of the AI model. In this case, the AI model may be trained using training data that includes medical images in which the medical instrument 140 is present and medical images in which the medical instrument 140 is absent. The AI model may be a U-net model, a Swim-Unet model, Segment Anything Model (SAM), or the like.

According to an embodiment, the medical imaging system 110 may generate the second medical image that includes inpainted data corresponding to the one or more regions of the first medical image that respectively correspond to the one or more medical instruments 140. For example, if the first medical image depicts a single medical instrument 140, then the medical imaging system 110 may generate the second medical image to include inpainted data in a single region that corresponds to the single medical instrument 140. Alternatively, if the first medical image depicts n medical instruments 140, then the medical imaging system 110 may generate the second medical image to include inpainted data in n regions that respectively correspond to the n medical instruments 140.

As further shown in FIG. 5, the process 500 may include displaying the second medical image (operation 540). For example, the medical imaging system 110 may display the second medical image. The second medical image may be substantially the same as the first medical image except that the second medical image does not depict the medical instrument 140. In this way, the clinician may more readily assess the entire region of interest because various anatomical structures are not occluded by the medical instrument 140.

The medical imaging system 110 may iteratively perform the operations of the process 500 as additional medical images of the region of interest are acquired during the interventional procedure. In this way, the displayed medical images depict the region of interest as not including the medical instrument 140 despite the medical instrument 140 actually being present in the region of interest.

Additionally, or alternatively, the medical imaging system 110 may perform similar operations with respect to a shadow, or shadows, caused by the medical instrument 140. For example, the medical imaging system 110 may determine a region of an acquired medical image that corresponds to a shadow caused by the medical instrument 140, and inpaint the region with inpainted data. In this way, the clinician may more readily assess the entire region of interest because various anatomical structures are not occluded by, or at least obfuscated by, the shadow of the medical instrument 140.

According to another embodiment, the medical imaging system 110 may generate the second medical image in which the medical instrument 140 is entirely absent. Restated, the second medical image might not depict any portion of the medical instrument 140. However, according to another embodiment, the medical imaging system 110 may generate the second medical image in which a portion of the medical instrument 140 is present. For example, the medical imaging system 110 may generate the second medical image may include a tip of the medical instrument 140, a particular amount of the medical instrument 140, an outline of the medical instrument 140, or the like.

According to another embodiment, the medical imaging system 110 may generate the second medical image to include a visual indicator corresponding to the location and/or direction of the medical instrument 140. For example, the visual indicator may be an icon, a geometric shape, an outline, or the like, that identifies a position and/or a direction of the medical instrument 140 in the second medical image. The medical imaging system 110 may determine the position and/or direction of the medical instrument 140 in the second medical image based on the determined region of the medical instrument 140, based on tracking data from the tracking system 120, or the like. In this way, the clinician may more readily assess the entire region of interest because various anatomical structures are not occluded by, or at least obfuscated by, the shadow of the medical instrument 140, but nonetheless may be apprised of the position and/or the direction of the medical instrument 140 in the region of interest based on the visual indicator.

Although FIG. 5 depicts particular operations and a particular sequence of operations, it should be understood that other embodiments may include different operations, more operations, fewer operations, or differently arranged operations.

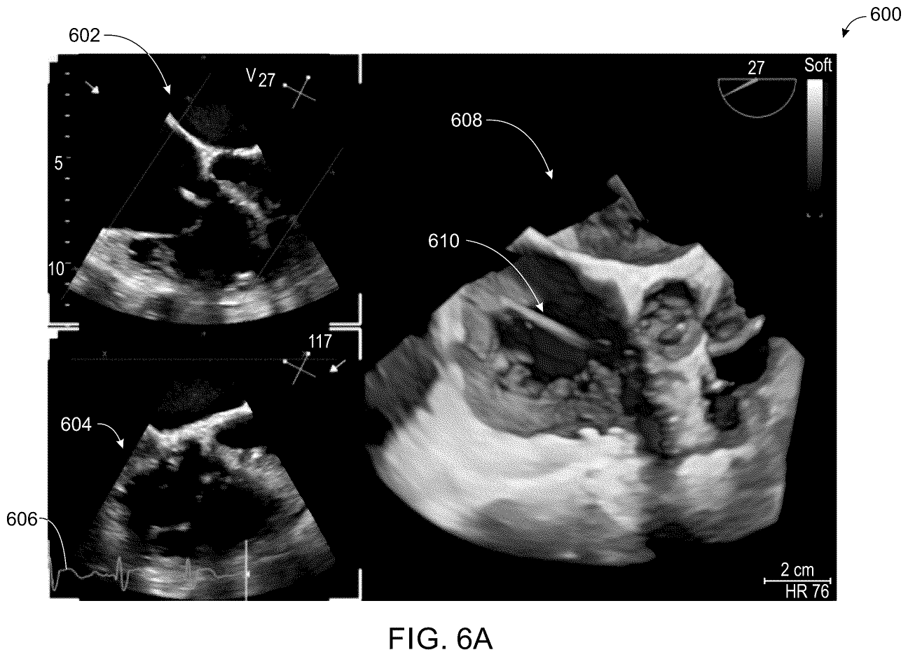

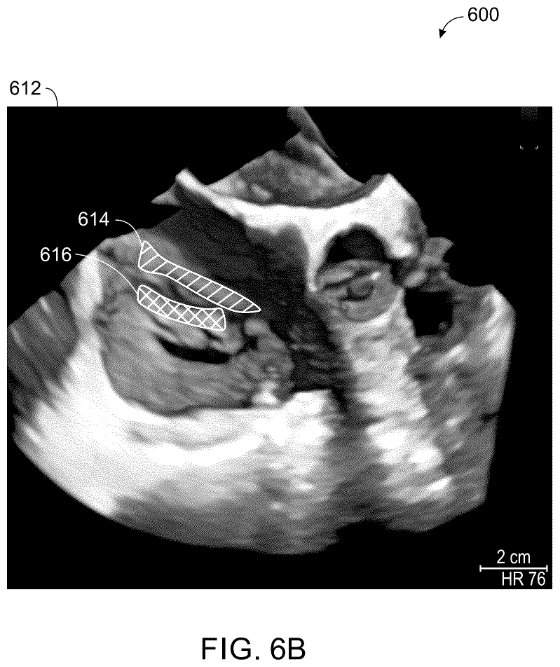

FIGS. 6A and 6B are diagrams of medical images. As shown in FIG. 6A, the medical imaging system 110 may display an ultrasound image 602 and an ultrasound image 604 corresponding to various view of a region of interest of a subject. Further, the medical imaging system 106 may display a visual indicator that identifies an ECG of the subject and that identifies a position of the ECG to which the ultrasound image 602 and the ultrasound image 604 correspond. As further shown in FIG. 6A, the medical imaging system 110 may display a volume-rendered ultrasound image 608 of the region of interest of the subject in which the medical instrument 140 is present. For instance, as shown by reference number 610, the volume-rendered image 608 may display the medical instrument 140 and a shadow caused by the medical instrument 140. For example, as shown in FIG. 6B, the medical imaging system 110 may display a volume-rendered image 612. As shown by reference umber 614, the medical instrument 140 may be present in the volume-rendered image 612 and, as shown by reference number 616, a shadow caused by the medical instrument 140 may be present in the volume-rendered image 614.

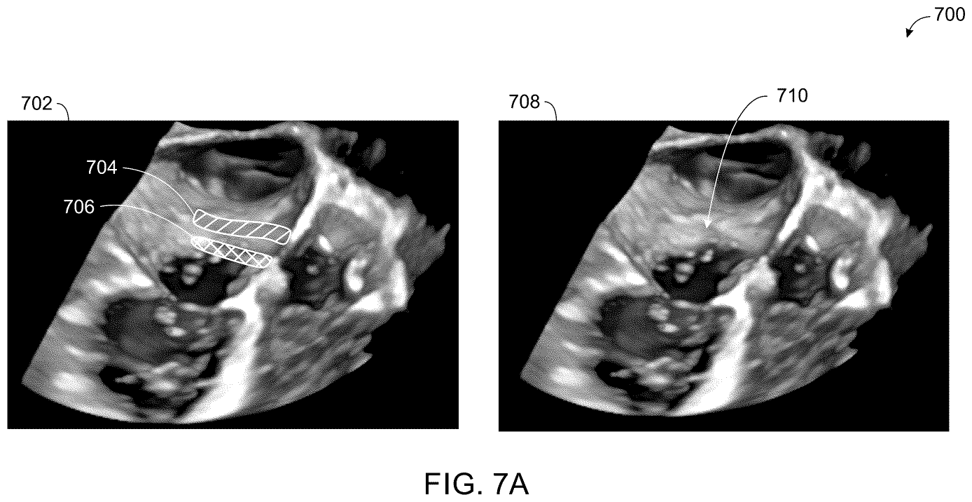

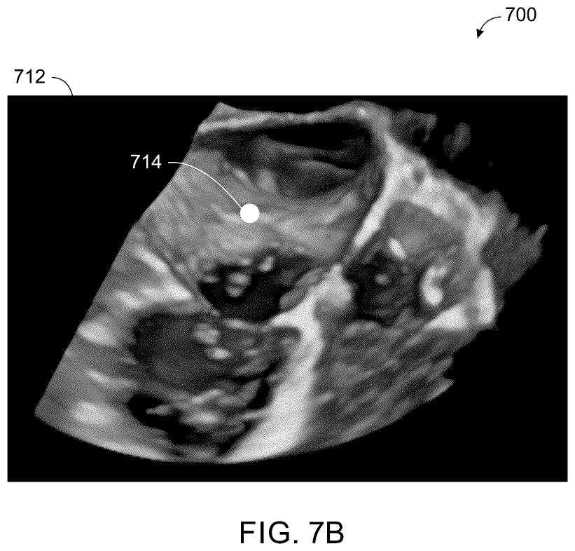

FIGS. 7A and 7B are diagrams of medical images. As shown in FIG. 7A, the medical imaging system 110 may acquire a first medical image 702 that depicts a region of interest of a subject and a medical instrument 140 in the region of interest of the subject. For example, as shown by reference number 704, the first medical image 702 may display the medical instrument 140 and, as shown by reference number 706, the first medical image 702 may display a shadow caused by the medical instrument 140. The medical imaging system 110 may perform the operations of the process 500, and generate and display the second medical image 708. As shown by reference number 710, the second medical image 708 does not depict the medical instrument 140 or the shadow of the medical instrument 140 in the region of interest despite the medical instrument 140 actually being physically present in the region of interest. In this way, the clinician may more readily assess the entire region of interest because various anatomical structures are not occluded by, or at least obfuscated by, the medical instrument 140 and the shadow of the medical instrument 140.

As shown in FIG. 7B, the medical imaging system 110 may display a second medical image 712 that does not depict the medical instrument 140 in the region of interest. As further shown in FIG. 7B, the medical imaging system 110 may display a visual indicator 714 that identifies a position of the medical instrument 140 in the second medical image 712. The medical imaging system 110 may track the position of the medical instrument 140, and update the display of the visual indicator 714 as the medical instrument 140 is moved throughout the region of interest of the subject.

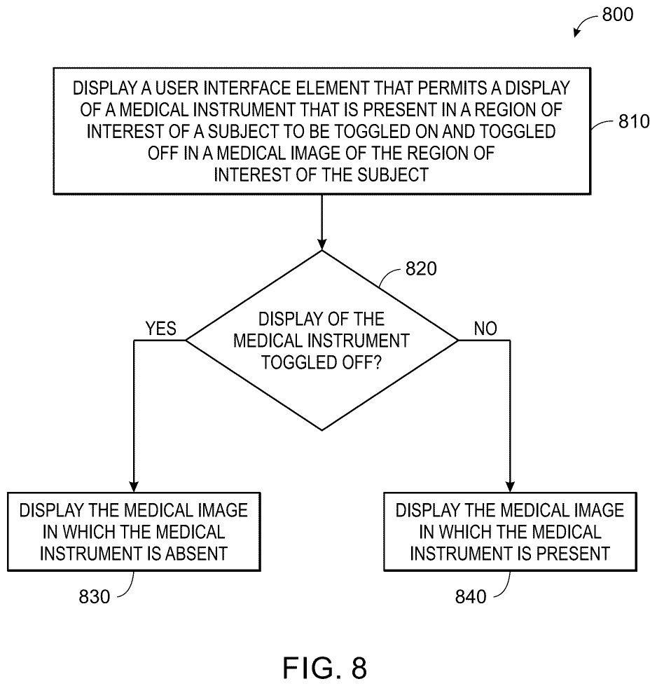

FIG. 8 is a flowchart of an example process for generating a medical image in which a medical instrument is absent or present based on whether the display of the medical instrument is toggled on or toggled off. According to an embodiment, the medical imaging system 110 may be configured to perform one or more operations of the process 800. Alternatively, another device may be configured to perform one or more operations of the process 800.

As shown in FIG. 8, the process 800 may include displaying a user interface element that permits a display of a medical instrument that is present in a region of interest of a subject to be toggled on and toggled off in a medical image of the region of the interest of the subject (operation 810). For example, the medical imaging system 110 may display a user interface element that permits a display of the medical instrument 140 that is present in a region of interest of a subject to be toggled on and toggled off in the medical image of the region of interest of the subject.

As further shown in FIG. 8, the process 800 may include determining whether the display of the medical instrument is toggled off (operation 820). For example, the medical imaging system 110 may determine whether the display of the medical instrument 140 is toggled off based on whether the clinician has interacted with the user interface element to toggle the display off.

As further shown in FIG. 8, if the display of the medical instrument is toggled off (operation 820—YES), then the process 800 may include displaying the medical image in which the medical instrument is absent (operation 830). For example, the medical imaging system 110 may display the medical image in which the medical instrument 140 is absent based on determining that the display of the medical instrument is toggled off.

As further shown in FIG. 8, if the display of the medical instrument is toggled on (operation 820—NO), then the process 800 may include displaying the medical image in which the medical instrument is present (operation 840). For example, the medical imaging system 110 may display the medical image in which the medical instrument 140 is present based on determining that the display of the medical instrument is toggled on.

The medical imaging system 110 may independently toggle the display of one or more medical instruments 140 on and off. For example, a user may select all of the one or more medical instruments 140 to be toggled off. Alternatively, the user may select a subset of the one or more medical instruments 140 to be toggled off. In this case, the displayed medical image may depict some, but not all, of the medical instruments 140 included in the region of interest.

Although FIG. 8 depicts particular operations and a particular sequence of operations, it should be understood that other embodiments may include different operations, more operations, fewer operations, or differently arranged operations.

In light of the foregoing, some embodiments herein provide a medical imaging system 110 for generating medical images in which a medical instrument 140 is absent from a depicted region of interest despite the medical instrument 140 actually being physically present in the region of interest. In this way, some embodiments herein provide a technical improvement in the technical field of medical imaging by permitting the medical imaging system 110 to generate a more comprehensive and accurate medical image that is not obfuscated by the present of a medical instrument 140 and a shadow of the medical instrument 140. Further, in this way, some embodiments herein provide a technical improvement to the medical imaging system 110 and a user interface of the medical imaging system 110 by permitting the medical imaging system 110 to generate more comprehensive and visually informative medical images, and by permitting a clinician to selectively toggle the display of the medical instrument 140 on and off during an interventional procedure.

Some embodiments herein are described in connection with the usage of AI models. For example, according to an embodiment, the medical imaging system 110 may determine the region of the first medical image corresponding to the medical instrument 140 using an AI model. As another example, and according to another embodiment, the medical imaging system 110 may generate the inpainted data of the region corresponding to the medical instrument 140 using an AI model. The AI models may be trained during a training phase, deployed during a deployment phase, and monitored during a monitoring phase.

The training phase may include receiving and processing training data to generate a trained AI model. According to an embodiment, the training data may include medical images in which medical instruments are present and corresponding information identifying the regions corresponding to the medical instruments. According to another embodiment, the training data may include medical images in which medical instruments are present and corresponding medical images in which the medical instruments are absent and in which the regions corresponding to the medical instruments are inpainted. The training data may be generated, received, or otherwise obtained from internal and/or external resources. For example,

Generally, the AI models may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the AI modes. According to an embodiment, a portion of the training data may be withheld during training and/or used to validate the trained AI models.

For supervised learning processes, the training data may include labels or scores that may facilitate the training process by providing a ground truth. Training may proceed by feeding a training dataset into the AI model. The AI model may have variables set at initialized values (e.g., at random, based on Gaussian noise, based on pre-trained values, or the like). The AI model may generate an output. The output may be compared with the corresponding label or score (e.g., the ground truth), which may then be back-propagated through the AI model to adjust the values of the variables. This process may be repeated for a plurality of samples at least until a determined loss or error is below a predefined threshold. According to an embodiment, some of the training data may be withheld and used to further validate or test the trained AI model.

For unsupervised learning processes, the training data may not include pre-assigned labels or scores to aid the learning process. Instead, unsupervised learning processes may include clustering, classification, or the like, to identify naturally occurring patterns in the training data 1104. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the AI model.

When reinforcement learning is employed, an agent (e.g., an algorithm) may be trained to make a decision regarding the training data through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained AI model may be stored and subsequently applied by the medical imaging system 110 during the deployment phase. For example, during the deployment phase, the trained AI model executed by the medical imaging system 110 may receive input data. According to an embodiment, the input data may include a medical image in which the medical instrument 140 is present. The AI model may provide, as output data, a medical image in which the region of the medical instrument 140 is determined. According to another embodiment, the AI model may provide, as output data, a medical image in which the medical instrument 140 is absent and in which a region corresponding to the medical instrument 140 is inpainted.

During the monitoring phase, monitoring data may be analyzed along with the predicted output data and input data to determine an accuracy of the trained AI model. According to an embodiment, based on the analysis, the training phase may be iteratively implemented where values of one or more variables of the AI model may be adjusted to improve the accuracy of the AI model.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
a memory configured to store instructions; and
one or more processors configured to execute the instructions to:
acquire a first medical image, of a region of interest of a subject, in which a medical instrument is present in the region of interest of the subject;
determine a region of the first medical image corresponding to the medical instrument;
generate a second medical image in which the medical instrument is absent using first medical image data of the first medical image external to the region and inpainted data internal to the region; and
display the second medical image.

2. The system of claim 1, wherein the one or more processors are further configured to:
determine a position and/or a direction of the medical instrument in the first medical image,
wherein the generating the second medical image comprises generating the second medical image to include a visual indicator at the position and/or the direction.

3. The system of claim 1, wherein the one or more processors are further configured to:
receive a user input that identifies that the medical instrument is to be removed from the first medical image,
wherein the generating the second medical image comprises generating the second medical image based on the user input that identifies that the medical instrument is to be removed from the first medical image.

4. The system of claim 1, wherein the one or more processors are further configured to:
acquire a third medical image, of the region of interest of the subject, in which the medical instrument is absent in the region of interest of the subject; and
compare the first medical image and the third medical image,
wherein the determining the region of the first medical image corresponding to the medical instrument comprises determining the region of the first medical image corresponding to the medical instrument based on comparing the third medical image and the first medical image.

5. The system of claim 1, wherein the region is a first region, and wherein the one or more processors are further configured to:
determine a second region of the first medical image corresponding to a shadow the medical instrument; and
determine a second region of the first medical image corresponding to the shadow of the medical instrument,
wherein the generating the second medical image comprises generating the second medical image in which the medical instrument and the shadow of the medical instrument are absent using the first medical image data of the first medical image external to the first region and the second region and inpainted data internal to the first region and the second region.

6. The system of claim 1, wherein the one or more processors are further configured to:
acquire a third medical image, of the region of interest of the subject, in which the medical instrument is absent in the region of interest of the subject and that corresponds to a same time point of a cycle of the region of interest as the first medical image,
wherein the inpainted data corresponds to third medical image data of the third medical image.

7. The system of claim 1, wherein the first medical image depicts one or more medical instruments, and wherein the one or more medical instruments are absent in the second medical image.

8. A method comprising:
acquiring a first medical image, of a region of interest of a subject, in which a medical instrument is present in the region of interest of the subject;
determining a region of the first medical image corresponding to the medical instrument;
generating a second medical image in which the medical instrument is absent using first medical image data of the first medical image external to the region and inpainted data internal to the region; and
displaying the second medical image.

9. The method of claim 8, further comprising:
determining a position and/or a direction of the medical instrument in the first medical image,
wherein the generating the second medical image comprises generating the second medical image to include a visual indicator at the position and/or the direction.

10. The method of claim 8, further comprising:
receiving a user input that identifies that the medical instrument is to be removed from the first medical image,
wherein the generating the second medical image comprises generating the second medical image based on the user input that identifies that the medical instrument is to be removed from the first medical image.

11. The method of claim 8, further comprising:
acquiring a third medical image, of the region of interest of the subject, in which the medical instrument is absent in the region of interest of the subject; and
comparing the first medical image and the third medical image,
wherein the determining the region of the first medical image corresponding to the medical instrument comprises determining the region of the first medical image corresponding to the medical instrument based on comparing the third medical image and the first medical image.

12. The method of claim 8, wherein the region is a first region, and wherein the method further comprises:
determining a second region of the first medical image corresponding to a shadow the medical instrument; and
determining a second region of the first medical image corresponding to the shadow of the medical instrument,
wherein the generating the second medical image comprises generating the second medical image in which the medical instrument and the shadow of the medical instrument are absent using the first medical image data of the first medical image external to the first region and the second region and inpainted data internal to the first region and the second region.

13. The method of claim 8, further comprising:
acquiring a third medical image, of the region of interest of the subject, in which the medical instrument is absent in the region of interest of the subject and that corresponds to a same time point of a cycle of the region of interest as the first medical image,
wherein the inpainted data corresponds to third medical image data of the third medical image.

14. The method of claim 8, wherein the generating the second medical image comprises generating the second medical image using an artificial intelligence model that is trained using first training images in which the medical instrument is absent, and second training images in which the medical instrument is present.

15. A non-transitory computer-readable medium storing instructions that, when executed by one or more processors, cause the one or more processors to:
acquire a first medical image, of a region of interest of a subject, in which a medical instrument is present in the region of interest of the subject;
determine a region of the first medical image corresponding to the medical instrument;
generate a second medical image in which the medical instrument is absent using first medical image data of the first medical image external to the region and inpainted data internal to the region; and
display the second medical image.

16. The non-transitory computer-readable medium of claim 15, wherein the instructions further cause the one or more processors to:
determine a position and/or a direction of the medical instrument in the first medical image,
wherein the generating the second medical image comprises generating the second medical image to include a visual indicator at the position and/or the direction.

17. The non-transitory computer-readable medium of claim 15, wherein the instructions further cause the one or more processors to:
receive a user input that identifies that the medical instrument is to be removed from the first medical image,
wherein the generating the second medical image comprises generating the second medical image based on the user input that identifies that the medical instrument is to be removed from the first medical image.

18. The non-transitory computer-readable medium of claim 15, wherein the instructions further cause the one or more processors to:
acquire a third medical image, of the region of interest of the subject, in which the medical instrument is absent in the region of interest of the subject; and
compare the first medical image and the third medical image,
wherein the determining the region of the first medical image corresponding to the medical instrument comprises determining the region of the first medical image corresponding to the medical instrument based on comparing the third medical image and the first medical image.

19. The non-transitory computer-readable medium of claim 15, wherein the region is a first region, and wherein the instructions further cause the one or more processors to:
determine a second region of the first medical image corresponding to a shadow the medical instrument;
determine a second region of the first medical image corresponding to the shadow of the medical instrument,
wherein the generating the second medical image comprises generating the second medical image in which the medical instrument and the shadow of the medical instrument are absent using the first medical image data of the first medical image external to the first region and the second region and inpainted data internal to the first region and the second region.

20. The non-transitory computer-readable medium of claim 15, wherein the instructions further cause the one or more processors to:
acquire a third medical image, of the region of interest of the subject, in which the medical instrument is absent in the region of interest of the subject and that corresponds to a same time point of a cycle of the region of interest as the first medical image,
wherein the inpainted data corresponds to third medical image data of the third medical image.
